(19) United States
(12) Patent Application Publication
(10) Pub. No.: US 2002/0106625 A1
(43) Pub. Date: Aug. 8, 2002

(54) Bioreactor for generating functional cartilaginous tissue

(76) Inventors: Clark T. Hung, Ardsley, NY (US); Gerald H. Aouni-Ateshian, New York, NY (US); Robert L. Mauck, New York, NY (US); Michael A. Soltz, Berkeley, CA (US); Wilmot B. Valhmu, Middleton, WI (US); Changbin Wang, New York, NY (US); Van C. Mow, Briaroliff Manor, NY (US)

Correspondence Address: William H Dippert, Cowan Liebowitz & Latman, 1133 Avenue of the Americas, New York, NY, 10036-6799, US

(21) Appl. No.: 10/049,761

(22) PCT Filed: Mar. 12, 2001
(86) PCT No.: PCT/US01/07815

(51) Int. Cl. C12M 3/00; C12N 5/00

(52) U.S. Cl. 435/1.1; 435/395; 435/289.1

(57) ABSTRACT

A bioreactor generates load-bearing cartilaginous or fibro-cartilaginous tissue by applying hydrostatic pressure and/or deformational loading to scaffolds seeded with chondrocytes and/or other cells. A scaffold may be shaped to reproduce the geometry of all or part of a load bearing articular surface or defect as acquired from a database or patient-specific geometry data. Optionally a scaffold can be attached to a substrate which promotes integration of this tissue construct with the underlying bone of the patient joint. In the bioreactor, ambient hydrostatic pressure and scaffold deformational loading can be prescribed with any desired waveform, using magnitudes which prevail in diarthrodial joints. The loading platen, permeable or impermeable, may conform to all or part of the scaffold surfaces.

### FIELD OF THE INVENTION

This invention is directed to a bioreactor for generating functional cartilaginous tissue. More particularly, this invention is directed to a bioreactor for producing functional, load-bearing cartilaginous tissue from cell-seeded scaffolds subjected to applied environmental hydrostatic pressurization and scaffold deformational loading at physiologic levels.

### BACKGROUND OF THE INVENTION

Introduction to Articular Cartilage: Articular cartilage serves as the load-bearing material of joints, with excellent friction, lubrication and wear characteristics (Mow V C, Ateshian G A., Ratcliffe A: Anatomic form and biomechanical properties of articular cartilage of the knee joint. In: Biology and biomechanics of the traumatized synovial joint: the knee as a model: AAOS Symposium, ed by G A M Finerman and F R Noyes, 1992). It is a white, dense, connective tissue, from 1 to 7 mm thick, that covers the bony articulating ends inside the joint It consists of two phases, a solid organic matrix (50% mass by dry weight collagen fibrils and 20-30% mass by dry weight proteoglycan macromolecules) (Clarke IC: Surface characteristics of human articular cartilage-a scanning electron microscope study. J Anat 108:23-30, 1971; Eyre D R: Collagen: Molecular diversity in the body's protein scaffold. Science 207:1315-1322, 1980; Fosang A J and Hardingham T E: Matrix proteoglycans. In: Extracellular Matrix, ed by W D Comper, Amsterdam, Harwood Academic Pubs., 1996; Muir H, Bullough P G, Maroudas A: The distribution of collagen in human articular cartilage with some of its physiological implications. J Bone Jt Surgery 52B:554-563, 1970; Muir I H M: The chemistry of the ground substance of joint cartilage. In: The Joints and Synovial Fluid, vol 2, ed by L Sokoloff, New York, Academic Press, 1980, pp 27-94) and a mobile interstitial fluid phase (predominately water) (Lipshitz H, Etheredge R, Glimcher M J: Changes in the hexosamine content and swelling ratio of articular cartilage as functions of depth from the surface. J Bone Jt Surg 58A:1149-1153, 1976; Maroudas A: Physicochemical properties of articular cartilage. In: Adult Articular Cartilage, ed by M A R Freeman, Kent, UK, Pitman Medical, 1979, pp 215-290). Aggregates of proteoglycans are important in the development and maintenance of cartilage. Expression of the genes for aggrecan and link protein, two major components of the aggregates, can be modulated by mechanical forces (Bachrach N M, Valhmu W B, Stazzone E, Ratcliffe A, Lai W M, Mow V C: Changes in proteoglycan synthesis of chondrocytes in articular cartilage are associated with the time dependent changes in their mechanical environment. J Biomech 28:1561-1569, 1995; Guilak F, Meyer B C, Ratcliffe A, Mow V C: The effects of matrix compression on proteoglycan metabolism in articular cartilage explants. Osteoarthritis Cartilage 2:91-101, 1994; Sah R L Y, Doong J-Y H, Grodzinsky A J, Plaas A H K, Sandy J D: Effects of compression on the loss of newly synthesized proteoglycans and proteins from cartilage explants. Arch Biochem Biophys 286:20-29, 1991; Sah R L Y, Kim Y J, Doong J-Y H, Grodzinsky A J, Plaas A H K, Sandy J D: Biosynthetic response of cartilage explants to dynamic compression. J Orthop Res 7:619-636, 1989; Valhmu W B, Stazzone E J, Bachrach N M, Saed-Nejad F, Fischer S G, Mow V C, Ratcliffe A: Load-controlled compression of articular cartilage induces a transient stimulation of aggrecan gene expression. Arch Biochem Biophys 353:29-36, 1998) as well as chemical stimuli (growth factors and cytokines). More recently, Mauck et al. (Mauck R L, Soltz M A, Wang C C-B, Wong D D, Chao P-H G, Valhmu W B, Hung, C. T. and Ateshian G A. Functional tissue engineering of articular cartilage through dynamic loading of chondrocyte-seeded agarose gels. J Biomech Eng 2000;122:252-260) demonstrated that physiologic deformational loading of chondrocyte-seeded agarose disks significantly enhanced matrix elaboration (proteoglycans and collagen) and the development of functional cartilage tissue properties. Because of its polyanionic glycosaminoglycan chains, the proteoglycan creates a large osmotic pressure that draws water into the tissue (Buschmann M D and Grodzinsky A J: A molecular model of proteoglycan-associated electrostatic forces in cartilage mechanics. J Biomech Engng 117:170-192, 1995; Lai W M, Hou J S, Mow V C: A triphasic theory for the swelling and deformational behaviors of articular cartilage. J Biomech Eng 113:245-258, 1991; Linn F C and Sokoloff L: Movement and composition of interstitial fluid of cartilage. Arthritis Rheum 8:481-494, 1965; Maroudas A: Physicochemical properties of articular cartilage. In: Adult Articular Cartilage, vol , ed by M A R Freeman, Kent, UK, Pitman Medical, 1979, pp 215-290) and expands the collagen network (Hardingham T E, Fosang A J, Dudhia J: Aggrecan, the chondroitin sulfate/keratan sulfate proteoglycan from cartilage. In: Articular Cartilage and Osteoarthritis, ed by K E Kuettner, et al., New York, Raven Press, 1992, pp 5-20). The balance between the osmotic swelling pressure of the proteoglycans and the tension in the collagen fibers (Kempson G E: Mechanical properties of articular cartilage. In: Adult Articular Cartilage, ed by M A R Freeman, Kent, England, Pitman Medical, 1979, pp 333-414) results in a highly specialized connective tissue which is well suited to bearing compressive load. Thus, the biomechanical properties of articular cartilage are dependent on the integrity of the collagen network and on maintenance of a high proteoglycan content within the matrix. Chondrocytes comprise less than 10% of the tissue volume (Stockwell R S: Biology of Cartilage Cells, Cambridge, Cambridge Press, 1979) and maintain this matrix, synthesizing and secreting extracellular matrix, to balance extracellular degradation and matrix turnover.

Physiologic Loading-Cartilage Mechanics: The loading environment of diarthrodial joints is generally well understood. Various classical studies have reported the magnitude of physiologic loads acting across lower and upper extremity joints (Cooney W P and Chao E Y S: Biomechanical analysis of static forces in the thumb during hand functions. J Bone Joint Surg 59-A:27-36, 1977; Paul J P: Forces transmitted by joints in the human body. Proc Instn Mech Engrs 181 (3J):8, 1967; Poppen N K and Walker P S: Forces at the glenohumeral joint in abduction. Clin Orthop Rel Res 135:165-70, 1978; Rydell N: Forces in the hip joint: Part (II) Intravital measurements. In: Biomechanics and Related Bio-Engineering Topics, ed by R M Kenedi, Oxford, Pergamon Press, 1965, pp 351-357). In general, it has been found that the peak magnitudes of such loads are a multiple factor of body weight (BW) in the lower extremities, e.g., 2.5 to 4.9 BW in the hip during walking (Armstrong C G, Bahrani A S, Gardner D L: In vitro measurement of articular cartilage deformations in the intact human hip joint under load. J Bone Jt Surg 61A((5)):744-755, 1979; Paul J P: Forces transmitted by joints in the human body. Proc Instn mech Engrs 181 (3J):8, 1967; Rydell N: Forces in the hip joint: Part (II) Intravital measurements. In: Biomechanics and Related Bio-Engineering Topics, ed by R M Kenedi, Oxford, Pergamon Press, 1965, pp 351-357) and 3.4 BW in the knee (Paul J P: Forces transmitted by joints in the human body. Proc Instn mech Engrs 181 (3J):8, 1967), or comparable to body weight in the upper extremities, e.g., 0.9 BW in the glenohumeral joint during abduction (Poppen N K and Walker P S: Forces at the glenohumeral joint in abduction. Clin Orthop Rel Res 135:165-70, 1978). Under normal circumstances, the loading duration of diarthrodial joints is generally cyclical and/or intermittent (Dillman C J: Kinematic analyses of running. In: Exercise and Sport Sciences Review, vol 3, ed by J H Wilmore, New York, Academic Press, 1975, pp. 193-218; Paul J P: Forces transmitted by joints in the human body. Proc Instn Mech Engrs 181 (3J):8, 1967), even for seemingly static activities such as standing or sitting, which involve back and forth shifting of the body weight to relieve loading of the joints; similarly, upper extremity activities rarely involve sustained static loading for durations in excess of a few minutes, though sustained dynamic (cyclical) loading may occur over a half-hour or more. Joint loads result in contact stresses at the articular surfaces, which have been extensively measured in the literature using various techniques (Ahmed A M: A pressure distribution transducer for in-vitro static measurements in synovial joints. ASME J Biomech Eng 105:309-314., 1983; Ahmed A M and Burke D L: In-vitro measurement of static pressure distribution in synovial joints—part I: Tibial surface of the knee. J Biomech Eng 105:216-225, 1983; Ahmed A M, Burke D L, Yu A: In-vitro measurement of static pressure distribution in synovial joints—part II: Retropatellar surface. J Biomech Eng 105: 226-236, 1983; Brown T D and Shaw D T: In vitro contact stress distributions in the natural human hip. J Biomechanics 16:373-384, 1983; Brown T D and Shaw D T: In vitro contact stress distribution on the femoral condyles. J Orthop Res 2:190-199, 1984; Fukubayashi T and Kurosawa H: The contact area and pressure distribution pattern of the knee. Acta Orthop Scand 51:871-879, 1980; Huberti H H and Hayes W C: Patellofemoral contact pressures. J Bone Joint Surg 66A: 715-724, 1984; Huberti H H and Hayes W C: Contact pressures in chondromalacia patellae and the effects of capsular reconstructive procedures. J Orthop Res 6:499-508, 1988; Kurosawa H, Fukubayashi T, Nakajima H: Load-bearing mode of the knee joint, physical behavior of the knee joint with or without menisci. Clin Orthop Rel Res 149:283-90, 1980; Manouel M, Pearlman HS, Belakhlef A, Brown T D: A miniature piezoelectric polymer transducer for in vitro measurement of the dynamic contact stress distribution. J Biomechanics 25:627-635, 1992; Singerman R J, Pedersen D R, Brown T D: Quantitation of pressure-sensitive film using digital image scanning. Experimental Mechanics March:99-105, 1987; Stormont T J, An K N, Morrey B F, Chao E Y: Elbow joint contact study: Comparison of techniques. J Biomechanics 18:329-336, 1985; Tencer A F, Viegas S F, Cantrell J, Chang M, Clegg P, Hicks C, O'Meara C, Williamson J B: Pressure distribution in the wrist joint. J Orthop Res 6:509-517, 1988). Typically, it has been found that activities of daily living produce mean contact stresses on the order of 2 MPa, while moderately strenuous activities result in mean contact stresses up to 6 MPa (Ahmed A M and Burke D L: In-vitro measurement of static pressure distribution in synovial joints—part I: Tibial surface of the knee. J Biomech Eng 105:216-225, 1983; Ahmed A M, Burke D L, Yu A: In-vitro measurement of static pressure distribution in synovial joints—part II: Retropatellar surface. J Biomech Eng 105: 226-236, 1983; Brown T D and Shaw D T: In vitro contact stress distributions in the natural human hip. J Biomechanics 16:373-384, 1983; Brown T D and Shaw D T: In vitro contact stress distribution on the femoral condyles. J Orthop Res 2:190-199, 1984; Huberti H H and Hayes W C: Patellofemoral contact pressures. J Bone Joint Surg 66A: 715-724, 1984; Huberti H H and Hayes W C: Contact pressures in chondromalacia patellae and the effects of capsular reconstructive procedures. J Orthop Res 6:499-508, 1988; Tencer A F, Viegas S F, Cantrell J, Chang M, Clegg P, Hicks C, O'Meara C, Williamson J B: Pressure distribution in the wrist joint. J Orthop Res 6:509-517, 1988); it has been estimated that the largest magnitude of mean contact stresses that may occur under non-traumatic conditions is about 12 MPa (Matthews L S, Sonstegard D A, Hanke J A: Load bearing characteristics of the patello-femoral joint Acta Orthop Scand 48:511-516, 1977), although in vivo measurements using instrumented endoprostheses (which relate indirectly to cartilage-on-cartilage contact) have reported contact stresses as high as 18 MPa (Hodge W A, Carlson K L, Fijan R S, Burgess R G, Riley P O, Harris W H, Mann R W: Contact pressures from an instrumented hip endoprosthesis. J Bone Joint Surg 71A:1378-1386, 1989). While in situ cartilage contact stresses have been extensively investigated, there is less information about in situ cartilage deformation. One radiographic cadaver study of hip joints reported a reduction of cartilage thickness by 20% or less in normal intact joints under physiologic loading of 5 BW (Armstrong C G, Bahrani A S, Gardner D L: In vitro measurement of articular cartilage deformations in the intact human hip joint under load. J Bone Jt Surg 61A((5)):744-755, 1979); another similar radiographic study has been reported on porcine joints (Wayne J S, Brodrick C W, Mukheljee N: Measurement of cartilage thickness in the articulated knee. Ann Biomed Engng 26(1):96-102, 1998). Ultrasound measurements of cartilage thickness in a cadaver hip experiment similarly demonstrated changes in cartilage thickness on the order of 10% or less, under 1.2 BW (Macirowski T, Tepic S, Mann R W: Cartilage stresses in the human hip joint. J Biomech Eng 116:10-18, 1994). In vivo magnetic resonance imaging (MRI) measurements of cartilage volumetric changes in the knees of human volunteers, prior and subsequent to strenuous activities, has demonstrated a reduction of 6% in cartilage volume (Eckstein F, Tieschky M, Faber S: In vivo quantification of patellar cartilage volume and thickness changes after strenuous dynamic physical activity—a magnetic resonance imaging study. Trans Orthop Res Soc 23:486, 1998). Theoretical contact analyses of biphasic cartilage layers under rolling or sliding motion have demonstrated that in a congruent joint, the cartilage layer thickness, decreases by 6% under a contact load of 1 BW (Ateshian G A and Wang H: A theoretical solution for the frictionless rolling contact of cylindrical biphasic articular cartilage layers. J Biomech 28:1341-1355, 1995).

Knowledge of the contact stresses at the articular surface is however insufficient to fully determine the state of stress inside cartilage. Recent joint contact and fluid pressure measurement studies (Ateshian G A, Lai W M, Zhu W B, Mow V C: An asymptotic solution for the contact of two biphasic cartilage layers. J Biomech 27:1347-1360, 1994; Ateshian G A Wang H: A theoretical solution for the frictionless rolling contact of cylindrical biphasic articular cartilage layers. J Biomech 28:1341-1355, 1995; Donzelli P S and Spilker R L: A contact finite element formulation for biological soft hydrated tissues. Comp Meth Appl Mech Engng 153:62-79, 1998; Kelkar R and Ateshian G A: Contact creep of biphasic cartilage layers: Identical layers. Journal of Applied Mechanics, ASME, In Press., 1999; Macirowski T, Tepic S, Mann R W: Cartilage stresses in the human hip joint. J Biomech Eng 116:10-18, 1994; Oloyede A and Broom N D: Is classical consolidation theory applicable to articular cartilage deformation? Clin Biomech 6: 206-212, 1991; Soltz M A and Ateshian G A: Experimental verification and theoretical prediction of cartilage interstitial fluid pressurization at an impermeable contact interface in confined compression. J Biomech 31:927-934, 1998; Van Der Voet A, Shrive N, Schachar N: Numerical modelling of articular cartilage in synovial joints—Poroelasticity and boundary conditions. In: Recent Advances in Computer Methods in Biomechanics & Biomedical Engineering, ed by J Middleton, G Pande, and K Williams, United Kingdom, Books & Journals International Ltd, 1993,) have confirmed the hypothesis that the interstitial water of articular cartilage pressurizes considerably when the joint is loaded (Linn F C and Sokoloff L: Movement and composition of interstitial fluid of cartilage. Arthritis Rheum 8:481-494, 1965; McCutchen C W: The frictional properties of animal joints. Wear 5:1-17, 1962; Mow V C Lai W M: Recent development in synovial joint biomechanics. SLAM Rev 22:275-313, 1980; Zarek J M and Edwards J: Dynamic considerations of the human skeletal system. In: Biomechanics and Related Bio-Engineering Topics, ed by R M Kenedi, Oxford, Pergamon Press, 1964, pp 187-293), contributing significantly to supporting the load transmitted across the articular layers. Various analyses have, suggested that the hydrostatic fluid pressure which develops in the interstitial water may contribute up to 90% or more of the contact stress measured at the articular surface (Ateshian G A, Lai W M, Zhu W B, Mow V C: An asymptotic solution for the contact of two biphasic cartilage layers. J Biomech 27:1347-1360, 1994; Ateshian G A and Wang H: A theoretical solution for the frictionless rolling contact of cylindrical biphasic articular cartilage layers. J Biomech 28:1341-1355, 1995; Kelkar R and Ateshian G A: Contact creep of biphasic cartilage layers: Identical layers. Journal of Applied Mechanics, ASME, In Press. 1999; Macirowski T, Tepic S, Mann R W: Cartilage stresses in the human hip joint. J Biomech Eng 116:10-18, 1994; Oloyede A and Broom N D: Stress-sharing between the fluid and solid components of articular cartilage under varying rates of compression. Connect Tissue Res 30:127-141, 1993). Thus, if a mean contact stress of 6.0 MPa is produced at the articular surfaces under physiologic loading, the cartilage interstitial fluid would pressurize to a mean value of 5.4 MPa approximately (depending on the joint congruence, cartilage properties, and loading rate). This pressurization occurs because the interstitial water attempts to squeeze out of the loaded region, but is impeded by the extremely low permeability of the collagen matrix. The significant contribution of interstitial fluid pressurization to the load support explains the findings (reported above) that in situ cartilage deformation is generally of moderate magnitude (˜20% of the thickness or less). While the interstitial fluid pressure has been shown to subside after several hours under purely static loading in a controlled in vitro laboratory environment (Grodzinsky A J, Lipshitz H, Glimcher M J: Electromechanical properties of articular cartilage during compression and stress relaxation. Nature 275, 1978; McCutchen C W: The frictional properties of animal joints. Wear 5:1-17, 1962; Mow V C, Gibbs M C, Lai W M, Zhu W B, Athanasiou K A: Biphasic indentation of articular carilage—Part II. A numerical algorithm and an experimental study. J Biomechanics 22:853-861, 1989; Mow V C Lai W M: Recent development in synovial joint biomechanics. SIAM Rev 22:275-313, 1980; Oloyede A and Broom N D: Is classical consolidation theory applicable to articular cartilage deformation? Clin Biomech 6: 206-212, 1991; Soltz M A and Ateshian G A: Experimental verification and theoretical prediction of cartilage interstitial fluid pressurization at an impermeable contact interface in confined compression. J Biomech, 31:927-934, 1998), recent studies have suggested that pressure subsidence and tissue consolidation are not likely to occur under in vivo physiologic conditions; therefore cartilage deformation caused by physiologic joint loading is always accompanied by significant interstitial fluid pressurization, i.e., cartilage deformation and interstitial fluid hydrostatic pressurization are synchronous and inseparable mechanisms in vivo (Soltz M A and Ateshian G A: Experimental verification and theoretical prediction of cartilage interstitial fluid pressurization at an impermeable contact interface in confined compression. Journal of Biomechanics, 31:927-934, 1998; Soltz M A and Ateshian G A: Measurement of cartilage fluid pressurization in confined compression cyclical loading. Advances in Bioengineering, ASME, BED39:23-24, 1998; Soltz M A and Ateshian G A: Interstitial fluid pressurization during confined compression cyclical loading of articular cartilage. Annals of Biomedical Engineering, 28(2):150-159, 2000). Furthermore, the normal physiologic loading environment can never be truly static; it is intermittent or cyclical, with loading duration ranging from fractions of a second (e.g., during gait (Dillman C J: Kinematic analyses of running. In: Exercise and Sport Sciences Review, Vol. 3, ed by J H Wilmore, New York, Academic Press, 1975, pp. 193-218; Paul J P: Forces transmitted by joints in the human body. Proc Instn mech Engrs 181 (3J):8, 1967) to a few minutes. Therefore, the normal loading environment of chondrocytes involves a combination of intermittent cyclical hydrostatic fluid pressurization and moderate scaffold deformation. If chondrocyte-seeded agarose disks are subjected to an appropriate combination of intermittently applied cyclical pressure and strain, the synthesis of a cartilage-like extracellular matrix will be optimally enhanced.

Mechanical Properties of Normal Cartilage: The mechanical properties of cartilage have been extensively investigated and reported, but it is important to appreciate that mechanical properties of any material are dependent on the accurate choice of constitutive relations which can suitably reproduce the experimentally determined response of that material, e.g., the stress-strain response. Some early studies of articular cartilage adopted constitutive relations which did not account for the presence of interstitial water, thus, the apparent modulus (a measure of stiffness) of cartilage reported in those studies may have been as high as 150 MPa (Kempson G E: Age-related changes in the tensile properties of human articular cartilage: a comparative study between the femoral head of the hip joint and the talus of the ankle joint. Biochim Biophys Acta 1075:223-230,1991; Kempson G E, Freeman M A, Swanson S A: Tensile properties of articular cartilage. Nature 220:1127-1128, 1968), since interstitial fluid pressurization contributes significantly (though impermanently) to load support. However, it is generally well accepted that various porous media theories and constitutive relations are more appropriate to describe the mechanical response of articular cartilage since they account for the presence of interstitial water (Frank E H and Grodzinsky A J: Cartilage electromechanics-II. a continuum model of cartilage electrokinetics and correlation with experiments. J Biomechanics 20(6):629-639, 1987; Lai W M, Hou J S, Mow V C: A triphasic theory for the swelling and deformational behaviors of articular cartilage. J Biomech Eng 113:245-258, 1991; McCutchen C W: The frictional properties of animal joints. Wear 5:1-17, 1962; Mow V C, Kuei S C, Lai W M, Armstrong C G: Biphasic creep and stress relaxation of articular cartilage in compression: theory and experiments. J Biomech Eng 102:73-84, 1980; Zarek J M and Edwards J: Dynamic considerations of the human skeletal system. In: Biomechanics and Related Bio-Engineering Topics, ed by R M Kenedi, Oxford, Pergamon Press, 1964, pp 187-293). From such studies, it has been demonstrated that the elastic modulus of articular cartilage ranges approximately from 0.2 to 1.4 MPa in compression (Ateshian G A, Warden W H, Kim J J, Grelsamer R P, Mow V C: Finite deformation biphasic material properties of bovine articular cartilage from confined compression experiments. J Biomechanics 30:1157-1164, 1997; Athanasiou K A, Rosenwasser M P, Buckwalter J A, Malinin T I, Mow V C: Interspecies comparison of in situ intrinsic mechanical properties of distal femoral cartilage. J Orthop Res 9:330-340, 1991; Frank E H and Grodzinsky A J: Cartilage electromechanics-II. a continuum model of cartilage electrokinetics and correlation with experiments. J Biomechanics 20(6):629-639, 1987; Lee R C, Frank E H, Grodzinsky A J, Roylance D K: Oscillatory compressional behavior of articular cartilage and its associated electromechanical properties. J Biomech Eng 103:280-292, 1981; Mow V C, Kuei S C, Lai W M, Armstrong C G: Biphasic creep and stress relaxation of articular cartilage in compression: theory and experiments. J Biomech Eng 102:73-84, 1980) and from 1 to 30 MPa in tension (Akizuki S, Mow V C, Muller F, Pita J C, Howell D S, Manicourt D H: Tensile properties of human knee joint cartilage: I. Influence of ionic concentrations, weight bearing and fibrillation on the tensile modulus. J Orthop Res 4:379-392, 1986; Grodzinsky A J, Roth V, Meyer E R, Grossman W, Mow V C: The significance of electromechanical and osmotic forces in the nonequilibrium swelling behavior of articular cartilage in tension. J Biomech Eng 103:221-231, 1981; Roth V and Mow V C: The intrinsic tensile behavior of the matrix of bovine articular cartilage and its variation with age. J Bone Jt Surg 62A: 1102-1117, 1980; Schmidt M B, Mow V C, Chun L E, Eyre D R: Effects of proteoglycan extraction on the tensile behaviour of articular cartilage. J Orthop Res 8:353-363, 1990; Woo S, Lubock P, Gomez M A, Jemmott G F, Kuei S C, Akeson W H: Large deformation nonhomogeneous and directional properties of articular cartilage in uniaxial tension. J Biomechanics 12:437-446, 1979; Woo S L-Y, Akeson W H, Jemmott G F: Measurements of nonhomogeneous directional mechanical properties of articular cartilage in tension. J Biomechanics 9:785-791, 1976). These elastic moduli represent the stiffness of the collagen-proteoglycan matrix of cartilage when the interstitial fluid pressure has subsided, which can be achieved experimentally after a long duration of static loading; thus they are often termed “equilibrium moduli.” The disparity between tensile and compressive properties and the ability to account for it in biomechanical models of cartilage remain important topics of research; recent studies (Ateshian G A and Soltz M A: Conewise linear elasticity mixture model for the analysis of tension-compression nonlinearity in articular cartilage. Trans Orthop Res Soc 12:158, 1999; Cohen B, Lai W, Mow V: A transversely isotropic biphasic model for unconfined compression of growth plate and chondroepiphysis. J Biomech Eng 120:491-496, 1998; Soulhat J, Buschmann M D, Shirazi-adl A: Non-linear cartilage mechanics in unconfined compression. J Biomechanics 23:226, 1998; Stamenovic D, McGrath C V, Bursa P M, Cooper J A, Eisenberg S R: A microstructural model of cartilage elasticity. Trans Orthop Res 23:223, 1998) suggest that it is possible to assess both tensile and compressive properties of cartilage from a combination of confined and unconfined compression tests of cylindrical disks.

The most commonly used constitutive law which describes how hydrostatic pressure gradients regulate the flow of interstitial fluid in cartilage is Darcy's law, whose material property is the tissue hydraulic permeability. Cartilage permeability has also been measured extensively, either through direct permeation experiments (Gu W Y, Rabin J, Lai W M, Mow V C: Measurement of streaming potential of bovine articular and nasal cartilage in a 1-D permeation experiment. Advances in Bioengineering ASME BED 31:49-50, 1995; Mansour J and Mow V C: The permeability of articular cartilage under compressive strain and at high pressures. J Bone Jt Surg 58A:509-516, 1976; Maroudas A, Bullough P, Swanson S A V, Freeman M A R: The permeability of articular cartilage. J Bone Jt Surg 50B:166-177, 1968), or indirectly from measuring the transient, flow-dependent response of cartilage under creep, stress-relaxation, or dynamic loading (Ateshian G A, Warden W H, Kim J J, Grelsamer R P, Mow V C: Finite deformation biphasic material properties of bovine articular cartilage from confined compression experiments. J Biomechanics 30:1157-1164, 1997; Athanasiou K A, Rosenwasser M P, Buckwalter J A, Malinin T I, Mow V C: Interspecies comparison of in situ intrinsic mechanical properties of distal femoral cartilage. J Orthop Res 9:330-340, 1991; Frank E H and Grodzinsky A J: Cartilage electromechanics-II. a continuum model of cartilage electrokinetics and correlation with experiments. J Biomechanics 20(6):629-639, 1987; Lai W M, Mow V C, Roth V: Effects of a nonlinear strain-dependent permeability and rate of compression on the stress behavior of articular cartilage. J Biomech Eng 103:221-231, 1981; Lee R C, Frank E H, Grodzinsky A J, Roylance D K: Oscillatory compressional behavior of articular cartilage and its associated electromechanical properties. J Biomech Eng 103:280-292, 1981; Mow V C, Kuei S C, Lai W M, Armstrong C G: Biphasic creep and stress relaxation of articular cartilage in compression: theory and experiments. J Biomech Eng 102:73-84, 1980). It has been observed that permeability is sensitive to the amount of cartilage deformation, or strain (Mansour J and Mow V C: The permeability of articular cartilage under compressive strain and at high pressures. J Bone Jt Surg 58A:509-516, 1976), thus some studies have provided a permeability function describing that relation (Holmes M H and Mow V C: The nonlinear characteristics of soft gels and hydrated connective tissues in ultrafiltration. J Biomechanics 23:1145-1156, 1990; Lai W M and Mow V C: Drag induced compression of articular cartilage during a permeation experiment. Biorheology 17:111-123, 1980). Most of the studies of cartilage permeability have reported consistent measurements of permeability, which ranges from 8×10⁻¹⁵ m⁴/N.s for normal cartilage under small strains, down to the order of 1×10⁻¹⁶ m⁴/N.s for cartilage under 50% compression. The mechanical properties of agarose are substantially different than those for articular cartilage.

Cartilage Tissue Engineering: Due to its avascular nature, cartilage exhibits a very limited capacity to regenerate and to repair. Moreover, it has been stated that the natural response of articular cartilage to injury is variable and, at best, unsatisfactory. The clinical need for improved treatment options for the numerous patients with cartilage injuries has motivated tissue engineering studies aimed at the in vitro generation of cartilage replacement tissues (or implants) using chondrocyte-seeded scaffolds (e.g., Chu, C. R., Coutts, R. D., Yoshioka, M., Harwood, F. L., Monosov, A. Z. and Amiel, D., 1995, “Articular cartilage repair using allogeneic perichondrocyte-seeded biodegradable porous polylactic acid (PLA): a tissue engineering study,” J. Biomed. Mater. Res. 29(9): 1147-1154; Dunkelman, N. S., Zimber, M. P., LeBaron, R. G., Pavelec, R, Kwan, M. and Purchio, A. F., 1995, “Cartilage production by rabbit articular chondrocytes on polyglycolic acid scaffolds in a closed bioreactor system,” Biotech. Bioeng. 46: 299-305., 1995; Freed, L. E., Langer, R., Martin, I., Pellis, N. R. and Vunjak-Novakovic, G., 1997, “Tissue engineering of cartilage in space,” Proc. Natl. Acad. Sci. 94: 13885-13890; Rahfoth, B., Weisser, J., Sternkopf, F., Aigner, T., von der Mark, K and Brauer, R., 1998, “Transplantation of allograft chondroctyes in agarose gel into cartilage defects in rabbits,” Osteoarthritis Cartilage 6(1): 50-65; Sittinger, M., Bujia, J., Minuth, W. W., Hammer, C. and Burmester, G. A, 1994, “Engineering of cartilage tissue using bioresorbable polymer carriers in perfusion culture,” Biomaterials 15(6):451456; Wakitani, S., Goto, T., Young, P, G., Mansour, J. M., Goldberg, V. M. and Caplan, A. 1., 1998, “Repair of large full-thickness articular cartilage defects with allograft articular chondrocytes embedded in a collagen gel.,” Tissue Eng. 4(4): 429-444). Although much of the tissue-engineered cartilage in existence has been successful in mimicking the morphological and biochemical appearance of hyaline cartilage, it is generally mechanically inferior to the natural tissue and requires a considerable culture period to develop. It is believed that the ability to restore the tissue's normal material properties will ameliorate the pain and suffering arising from osteoarthritis (OA), a debilitating disease which results in the erosion of diarthtrodial joint cartilage. Currently, OA affects 5% of the general population and 70% of the population over age 65 and costs nearly $8 billion annually in health care (Kuettner, K. E. and Goldberg, V. M. (1995): Osteoarthritic Disorders. American Academy of Orthopaedic Surgeons, Rosemont, Ill. preface p:xix.).

Given the lengthy period of time required for development of mechanical properties under conditions of static or free swelling culture, many researchers have sought to design bioreactors to speed the production of cartilage in vitro. A prevailing thought is that “sophisticated culture systems, involving precise control of media, mixing, shear force, and hydrodynamic pressure, are often necessary to culture chondrocytes successfully” (Ehrenreich, M., 1999, “Articular cartilage repair: tissue engineering's killer application?,” Techvest, LLC Equity Research Farndale, R. W., Sayers, C. A. and Barrett, A. J., 1982, “A direct spectrophotometric microassay for sulfated glycosaminoglycans in cartilage cultures,” Connect. Tissue Res. 9: 247-248). Malaviya and Nerem have adopted a parallel-plate flow chamber system to use fluid-induced shear stress as a modulator of chondrocyte activities for tissue engineering of cartilage (Malaviya, P., Hunter, C., Seliktar, D., Schreiber, R., Symons, K., Ratcliffe, A. and Nerem, R., 1998, “Fluid-induced shear stresses promote chondrocyte phenotype alteration,” Trans Orthop Res 23(1): 228; Malaviya, P. and Nerem, R. M., 1999, “Steady shear stress stimulates bovine chondrocyte proliferation in monolayer cultures,” Transactions of the Orthopaedic Research Society 24: 8;). Perfusion systems, rotating wall vessels and spinner flasks have also been adopted with some success (Freed, L. E., Vunjak-Novakovic, G. and Langer, R., 1993, “Cultivation of cell-polymer cartilage implants in bioreactors,” J. Cell. Biochem. 51: 257-264; Freed, L. E., Grande, D. A., Lingbin, Z., Emmanual, J., Marquis, J. C. and Langer, R., 1994, “Joint resurfacing using allograft chondrocytes and synthetic biodegradable polymer scaffolds,” J Biomed. Mater. Res. 28: 891-899; Freed, L. E., Marquis, J. C., Vunjak-Novakovic, G., Emmanual, J. and Langer, R., 1994, “Composition of cell-polymer cartilage implants,” Biotech. Bioeng. 43: 605-614; Dunkelman et al., 1995; Carver, S. E. and Heath, C. A., 1999, “Influence of intermittent pressure, fluid flow, and mixing on the regenerative properties of articular chondrocytes,” Bitechnol. Bioeng. 65(3): 274-281; Carver, S. E. and Heath, C. A., 1999, “Semi-continuous perfusion system for delivering interrmittent physiological pressure to regenerating cartilage,” Tissue Eng 5(1): 1-11; Davisson, T. H., Wu, F. J., Jain, D., Sah, R. L. and Ratcliffe, A. R., 1999, “Effect of perfusion on the growth of tissue engineered cartilage,” Trans. Orthop. Res. Soc. 45: 811). Polyglycolic acid (PGA) felts in a closed bioreactor system require 4-6 weeks for chondrocytes to synthesize a cartilage-like appearance (as reviewed by Ehrenreich, 1999). Bioresorbable scaffolds encapsulated by agarose were found to improve retention and accumulation of extracellular matrix proteins from chondrocytes in perfusion culture (Sittinger et al., 1994). Interestingly, many of these bioreactors seem to be generic or non-specific in that they foster tissue growth of many tissue types in culture (e.g., Goodwin, T. J., Jessup, J. M. and Wolf, D. A., 1992, “Morphologic differentiation of colon carcinoma cell lines HT-29 and HT-29KM in roating-wall vessels,” In vitro Cell. Dev. Biol. 28A: 47-60; Duke, P. J., Daane, E. L. and Montufar-Solis, D., 1993, “Studies of chondrogenesis in rotating systems,” J. Cell. Biochem. 51: 274-282; Goodwin, T. J., Schroeder, W. F., Wolf, D. A. and Moyer, M. P., 1993, “Rotating-wall vessel coculture of small intestine as a prelude to tissue modeling: aspects of simulated microgravity”; Spaulding, G. F., Jessup, J. M. and Goodwin, T. J., 1993, “Advances in cellular construction,” I Cell. Biochenm 51). Unlike the case of articular cartilage, mechanical loading may only play a minor role in the development and maintenance of other tissues. Therefore, it is desirable to create a bioreactor which is tailored specifically for cartilage tissue growth.

The mechanical environment is known to influence the phenotypic expression and activity of the chondrocytes within the matrix; immobilization is clearly detrimental to cartilage development and repair (e.g., Grumbles, R. M., Howell, D. S., Howard, G. A., et al., 1995, “Cartilage metalloproteases in disuse atrophy,” I Rheumatol, 43 (supplement): 146-8; Setton, L. A., Mow, V. C. and Howell, D. S., 1995, “Mechanical behavior of articular cartilage in shear is altered by transection of the anterior cruciate ligament,” J. Orthop. Res. 13(4): 473-82). Under normal conditions, chondrocytes are able to balance their synthetic and catabolic activities to maintain the integrity of articular cartilage in vivo. In view of this fact, we desired to restore aspects of the physiologic environment to articular chondrocytes in vitro with the rationale that a physiologic environment is paramount to producing a tissue that is able to perform the load-bearing and lubrication function of natural cartilage.

From the literature, only short-term (typically one week or less) deformational loading studies of cell-seeded scaffolds have been performed. In these studies, parameters such as cell deformation, cell signaling pathways and chondrocyte biosynthesis rates have been monitored (Lee, D. A. and Bader, D. L., 1995, “The development and characterisation of an in vitro system to study strain-induced cell deformation in isolated chondrocytes,” In vitro Cell Dev Biol Anim 31: 828-835; Lee, D. A. and Bader, D. L., 1997, “Compressive strains at physiological frequencies influence the metabolism of chondroctyes seeded in agarose,” J. Orthop. Res. 15: 181-188; Knight, M. M., Lee, D. A. and Bader, D. L., 1998, “The influence of elaborated pericellular matrix on the deformation of isolated articular chondrocytes cultured in agarose,” Biochem. Biophys. Acta 1405: 67-77; Lee, D. A., Frean, S. P., Lees, P. and Bader, D. L., 1998, “Dynamic mechanical compression influences nitric oxide production by articular chondrocytes seeded in agarose,” Biochem. Biophys. Res. Comm. 251: 580-585.). In a variation of the short term loading study, Buschmann and co-workers assessed the response of cell-seeded agarose disks cultured statically with time to short-term dynamic loading to assess biosynthetic activity of chondrocytes in a cell elaborated matrix and reported that cell biosynthetic activity in cultured cell-seeded agarose disks resemble that for articular cartilage. No studies are known to have investigated the efficacy of applied deformational loading in enhancing matrix elaboration in long-term cultures of chondrocyte-seeded scaffolds.

### OBJECTS OF THE INVENTION

It is an object of this invention to enhance matrix elaboration by chondrocytes seeded in three dimensional scaffolds in cultures subjected to optimized combinations of physiologic hydrostatic pressure and deformational loading at physiologic levels.

It is also an object of the invention to improve the functional (mechanical, electrical, chemical, biochemical) properties of chondrocyte-seeded three dimensional scaffolds cultivated under combinations of physiologic hydrostatic pressure and deformational loading over those developed under pressurization or deformational loading alone.

It is a further object of the invention to optimize the growth of cartilage in vitro by varying the daily duration and frequency of loading, and the magnitude of hydrostatic pressurization and deformational loading.

It is a yet further object of the invention to develop a bioreactor that comprises means for producing tissue in desired shapes wherein the shaped tissue conforms to a body part, a prosthesis, a cosmetic implant, or a defect to be filled. The latter will entail loading with platens that conform to all or part of the articular surface. The geometry of the platens can be obtained from a database or patient-specific geometry data.

These and other objects of the invention will become more apparent from the discussion below.

### SUMMARY OF THE INVENTION

A bioreactor is proposed for generation of load-bearing cartilaginous or fibro-cartilaginous tissue by applying hydrostatic pressure and/or deformational loading to scaffolds seeded with chondrocytes and/or other cells. Scaffolds may be shaped to reproduce the geometry of all or part of a load bearing articular surface or defect as acquired from a database or patient-specific geometry data A scaffold is optionally attached to a substrate (e.g., hydroxyapatite) which promotes integration of this tissue construct with the underlying bone of the patient joint. In the bioreactor, ambient hydrostatic pressure and scaffold deformational loading can be prescribed with any desired waveform, using magnitudes which prevail in diarthrodial joints. The loading platen, permeable or impermeable, may conform to all or part of the scaffold surfaces. This bioreactor maintains the sterility necessary for the production of bioengineered tissue constructs. This invention differs from the closest prior art in that: it provides simultaneous hydrostatic pressure and tissue deformation in a physiologic range, utilizes loading platens which can conform to a designated shape of the tissue construct, and provides for an attachment to promote integration with the underlying bone. Control of matrix strain rather than stress or load is specifically chosen to protect cells from being subjected to levels of deformation that may be detrimental to cell viability and tissue growth during applied loading.

According to the invention, the physiologic combination of intermittent hydrostatic pressure and deformational loading of cell-seeded scaffolds results in optimal generation of tissue with functional properties and biochemical composition similar to articular cartilage. An apparatus useful according to the invention is a bioreactor comprising a growth chamber for housing cultured cells, a cell-seeded three-dimensional scaffold, optionally integrated with a substrate that promotes bony ingrowth for attachment to underlying bone, means for applying hydrostatic pressure and means for applying deformational loading. The bioreactor applies hydrostatic pressure at a level of from about 0 to about 18 MPa, with a preferred range of from about 0 to about 6 MPa. In addition, the bioreactor applies deformation of from about 0 to about 50% of a representative thickness of the cell-seeded scaffold, with a preferred range of 0 to about 20%.

The scaffold in the bioreactor supports the growth of a 3-dimensional cell culture. The scaffold can be bioresorbable. Preferably the substrate is conducive to bony ingrowth.

The invention also provides a method for producing functional cartilaginous tissue from a cell-seeded scaffold or a cell-seeded scaffold integrated with an osteoconductive and/or osteoinductive substrate. The method comprises the steps of (a) inoculating chondrocytes or chondroprogenitors into a scaffold or a scaffold integrated with an osteoinductive substrate; (b) placing cell-seeded scaffold or cell-seeded scaffold integrated with an osteoinductive substrate into a bioreactor; (c) filling said bioreactor with liquid growth medium; (d) applying hydrostatic pressurization and/or deformational loading to the cell-seeded scaffold or cell-seeded scaffold integrated with an osteoinductive substrate; and (e) culturing said stressed cell-seeded scaffold or cell-seeded scaffold integrated with an osteoinductive substrate for a time sufficient to produce functional cartilaginous tissue.

The physiologically loaded cell-seeded scaffold grown according to this method displays enhanced maintenance of the chondrocyte phenotype. In addition, the cells produce a cartilage-like extracellular matrix.

As used herein, “bioresorbable” means biodegradable in cell culture or in the body of an artificial cartilage transplant recipient.

As used herein, “chondrocyte” means a cartilage cell. Chondrocytes are found in various types of cartilage, e.g., articular (or hyaline) cartilage, elastic cartilage, and fibrocartilage.

As used herein, “substrate” means a supporting structure to which the cell-seeded scaffold is anchored and which is conducive to bony ingrowth.

As used herein, “scaffold” means a three-dimensional, porous, cell culture-compatible structure, throughout which cultured mammalian cells can be seeded so as to form a 3-dimensional culture.

As used herein, “hydrostatic pressure” means a fluid-borne compressive isotropic stress (i.e., equal in all directions) acting on cultured cells.

As used herein, “deformational loading” means a relative change in one or more of the characteristic dimensions of the cell-seeded scaffold.

As used herein, “stem cell” means an undifferentiated cell with the potential to mature into the specialized cells characterizing a particular tissue.

As used herein, “transdifferentiation” means the change of a differentiated cell from one phenotype, e.g., myoblast or fibroblast, into another phenotype, e.g., a chondrocyte.

As used herein, “functional properties” means possessing the mechanical, electrical, chemical and biochemical properties of cartilaginous tissues—the properties that permit cartilage to perform and maintain its load-bearing capacity.

Unless otherwise defined, all technical and scientific terms used herein have the same meaning as commonly understood by one skilled in the art of cell culturing techniques and biomechanics. Although materials and methods similar or equivalent to those described herein can be used in the practice or testing of the invention, the preferred methods and materials are described below. All publications, patent applications, patents and other references mentioned herein are incorporated by reference. In addition, the materials, methods, and examples are illustrative only and not intended to be limiting.

Other advantages and features of the invention will be apparent from the detailed description, and from the claims.

### BRIEF DESCRIPTION OF THE DRAWINGS

FIG. 1 is a schematic representation of a bioreactor vessel according to the invention;

FIG. 2 is a schematic diagram of the operation of the bioreactor according to the invention;

FIGS. 3 and 4 are each a perspective view of a bioreactor according to the invention;

FIG. 5 is a view of the interior of a bioreactor according to the invention with the top removed; and

FIGS. 6 to 13 reflect the steps utilized in the creation of a scaffold construct useful in the bioreactor.

### DETAILED DESCRIPTION OF THE INVENTION

According to the invention functional cartilaginous tissue with appropriate form and function for in vivo implantation can be created by selectively stimulating the growth and differentiated function of chondrocytes (i.e., proteoglycan and collagen synthesis) through optimization of the in vitro culture environment. Cells are inoculated into a three-dimensional scaffold, and grown in culture to form a living cartilaginous material. The cells may comprise chondrocytes, chondroprogenitors, with or without additional cells and/or elements described more fully herein. These cells may be fetal or adult in origin, and may be derived from convenient sources such as cartilage, skin, etc. Such tissues and/or organs can be obtained by appropriate biopsy or upon autopsy; cadaver organs may be used to provide a generous supply of cells and elements. Alternatively, umbilical cord and placenta tissue or umbilical cord blood may serve as an advantageous source of fetal-type stem cells, e.g., chondroprogenitor cells for use in the three-dimensional system of the invention.

Cells can be inoculated into the scaffold to form a “generic” living tissue for culturing any of a variety of cells and tissues. However, in certain instances, it may be preferable to use a “specific” rather than “generic” system, in which case cells and elements can be obtained from a particular tissue, organ, or individual. For example, where scaffold is to be used for purposes of transplantation or implantation in vivo, it may be preferable to obtain the cells and elements from the individual who is to receive the transplant or implant. This approach might be especially advantageous where immunological rejection of the transplant and/or graft versus host disease is likely.

Once inoculated into the 3-dimensional scaffold, the cells will proliferate in the scaffold and form the living tissue which can be used in vivo. The three-dimensional living tissue will sustain active proliferation of the culture for long periods of time.

In this application, the three-dimensional scaffold is cultured in a bioreactor to produce cartilage tissue constructs possessing functional properties, under environmental conditions which are typically experienced by native cartilage tissue. The functional properties and rate of production of cartilage in the three-dimensional culture are significantly improved by the application of combined intermittent cyclical pressurization and deformational loading.

The three-dimensional cultures may also be used in vitro for testing the effectiveness or cytotoxicity of pharmaceutical agents, and screening compounds.

The bioreactor maintains an adequate supply of nutrients. Maintaining an adequate supply of nutrients to chondrocyte cells throughout a replacement cartilage tissue construct is extremely important as matrix elaborates in the scaffold.

The three-dimensional scaffold may be of any material and/or shape that allows cells to attach to or be encapsulated in it (or can be modified to allow cells to attach to it or be encapsulated in it). A number of different materials may be used to form the matrix, including but not limited to: hydrogels (e.g., agarose and alginate), nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), nitrocellulose, cotton, polyglycolic acid (PGA), collagen (in the form of sponges, braids, or woven threads, etc.), catgut sutures, cellulose, gelatin, or other naturally occurring biodegradable materials or synthetic materials, including, for example, a variety of polyhydroxyalkanoates. Any of these materials may be woven into a mesh, for example, to form the three-dimensional scaffold. Certain materials, such as nylon, polystyrene, etc. are poor substrates for cellular attachment. When these materials are used as the three-dimensional scaffold, it is advisable to pre-treat the matrix prior to inoculation of cells in order to enhance their attachment to the scaffold. For example, prior to inoculation with cells, nylon matrices could be treated with 0.1M acetic acid and incubated in polylysine, PBS, and/or collagen to coat the nylon. Polystyrene could be similarly treated using sulfuric acid. Where the cultures are to be maintained for long periods of time or cryopreserved, non-degradable materials such as nylon, dacron, polystyrene, polyacrylates, polyvinyls, teflons, cotton, etc., may be preferred. A convenient nylon mesh which could be used in accordance with the invention is Nitex, a nylon filtration mesh having an average pore size of 210 microns and an average nylon fiber diameter of 90 microns (#3-210/36 Tetko, Inc., New York).

Where the three-dimensional culture is itself to be implanted in vivo, it may be preferable to use biodegradable matrices such as agarose, polyglycolic acid, a polymer supplemented with a hydrogel (such as polyglycolic acid encapsulated in agarose), catgut suture material, collagen, or gelatin, for example. Agarose is commonly sterilized in preparation for long-term in vitro culture by autoclaving or sterile filtration. Cells comprising chondrocytes, chondroprogenitors, with or without other cells and elements described below, are inoculated into the scaffold.

Cells such as chondrocytes may be derived from articular cartilage, costal cartilage, etc. which can be obtained by biopsy (where appropriate) or upon autopsy. Fetal cells, including chondroprogenitors, may be obtained from umbilical cord or placenta tissue or umbilical cord blood. Such fetal cells can be used to prepare a “generic” cartilaginous tissue. However, a “specific” cartilaginous tissue may be prepared by inoculating the three-dimensional scaffold with cells derived from a particular individual who is later to receive the tissues grown in culture in accordance with the three-dimensional system of the invention.

Cells may also be isolated from human umbilical cords (33-44 weeks). Fresh tissues may be minced into pieces and washed with medium or snap-frozen in liquid nitrogen until further use. The umbilical tissues may be disaggregated as described above.

Once the tissue has been reduced to a suspension of individual cells, the suspension can be fractionated into subpopulations from which the desired cells and/or elements can be obtained. This also may be accomplished using standard techniques for cell separation including but not limited to cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counter-streaming centrifugation), unit gravity separation, counter current distribution, electrophoresis and fluorescence-activated cell sorting.

The isolation of chondrocytes, chondroprogenitors and other cells may, for example, be carried out as follows: fresh tissue samples are thoroughly washed and minced in Hanks balanced salt solution (HBSS) in order to remove serum. The minced tissue is incubated from 1-12 hours in a freshly prepared solution of a dissociating enzyme such as hyaluronidase and collagenase. After such incubation, the dissociated cells are suspended, pelleted by centrifugation and plated onto culture dishes. All fibroblasts will attach before other cells, therefore, appropriate cells can be selectively isolated and grown. The isolated cells can then be grown to confluency, lifted from the confluent culture and inoculated onto the three-dimensional scaffold (see, Naughton et al., 1987, J. Med. 18(3&4):219-250). Inoculation of the three-dimensional scaffold with a high concentration of cells, e.g., approximately 10⁶ to 5×10⁷ cells/ml, will result in the establishment of the three-dimensional tissue in shorter periods of time.

In addition to chondrocytes or chondroprogenitors, other cells may be added to form the three-dimensional scaffold required to support long term growth in culture. For example, other cells found in loose connective tissue may be inoculated onto the three-dimensional scaffold along with chondrocytes. Such cells include, but are not limited to, endothelial cells, pericytes, macrophages, monocytes, plasma cells, mast cells, adipocytes, etc. These cells may readily be derived from appropriate organs including umbilical cord or placenta or umbilical cord blood using methods known in the art such as those discussed above.

Again, where the cultured cells are to be used for transplantation or implantation in vivo it is preferable to obtain the cells from the patient's own tissues. The growth of cells on the three-dimensional scaffold may be further enhanced by incorporating proteins (e.g., RGDs, collagens, elastic fibers, reticular fibers) glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials into the scaffold.

After inoculation of the cells, the three-dimensional scaffold should be incubated in an appropriate nutrient medium. Many commercially available media such as DMEM, RPMI 1640, Fisher's Iscove's, McCoy's, and the like may be suitable for use. The culture should be “fed” periodically to remove the spent media, depopulate released cells, and add fresh media. The concentration of agonists may be adjusted during these steps. In chondrocyte cultures, proline, a non-essential amino acid, and ascorbate are also included in the cultures.

Bioreactor

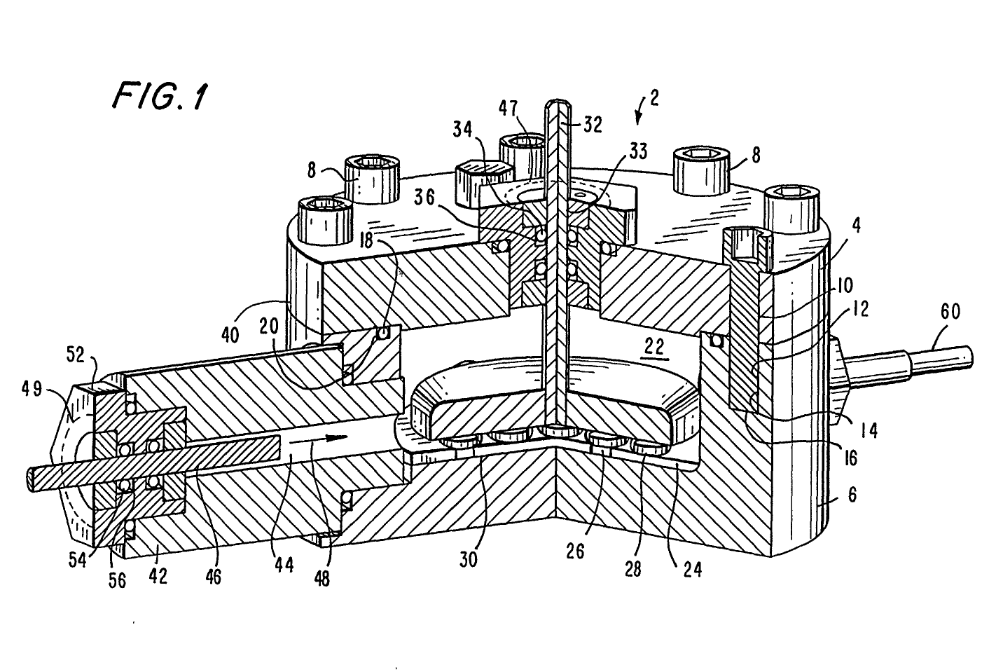

A schematic of one embodiment of the bioreactor useful according to the invention is shown in FIG. 1. The bioreactor vessel 2 comprises an upper member or vessel cap 4 and a lower member 6, secured together by bolts 8. Preferably each bolt 8 fits through an opening 10 in vessel cap 4, and the outer cylindrical surface 12 of each bolt 8 has threads that engage mating threads 14 in each tapped hole 16. Sealing is effected by an O-ring 18 in a groove 20.

Within chamber 22 of vessel 2 an agarose template 24 has indentations or wells 26 that contain cell-seeded agarose disks 28. These wells prevent shifting of the disks during loading or transport. A compression loading platen 30 is rigidly attached to a actuator rod 32 that extends through opening 33 in vessel cap 4 to a displacement actuator device (not shown). O-rings 34 in grooves 36 provide sealing around actuator rod 32.

A lateral surface 40 of lower member 6 has removably engaged thereto a hydraulic pressure assembly 42 having a lumen or piston chamber 44. A hydraulic pressure control rod or piston 46 extends within lumen 44, the proximal end of pressure control rod 46 being operatively connected to a displacement actuator device (not shown). When pressure control rod 46 is moved in the distal direction, as shown by arrow 48, the hydrostatic pressure in chamber 22 increases. Pressure assembly 42 comprises an end member 52 through which pressure control rod 46 passes. O-rings 54 in grooves 56 provide sealing.

Another portion of lateral surface 40 of lower chamber 6 comprises a pressure transducer 60 for measurement of the hydrostatic pressure within chamber 22. Transducer 60 is operatively, e.g., mechanically or electrically, connected to a pressure read-out (not shown).

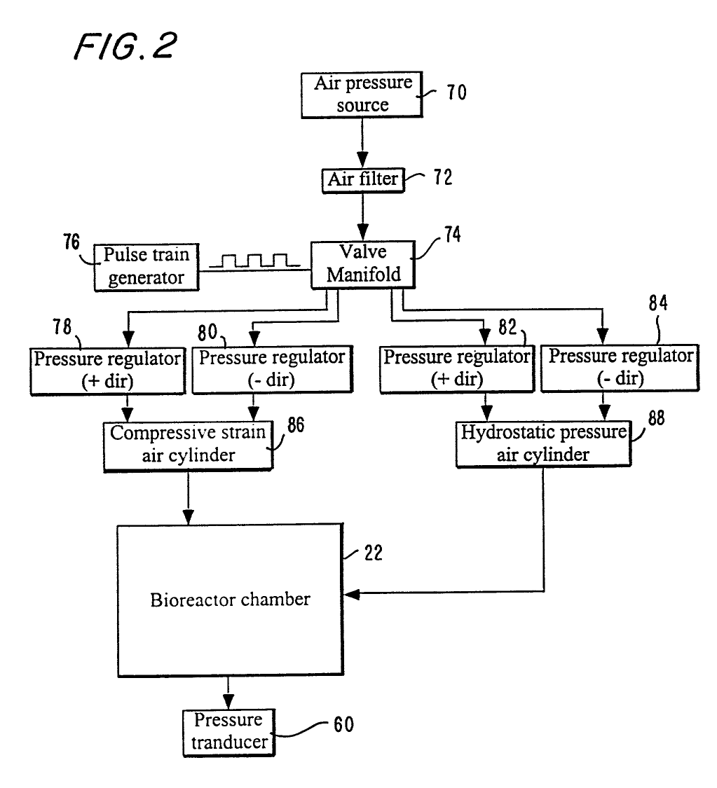

FIG. 2 represents a schematic of the operation of a chamber of FIG. 1 according to the invention. Air from air pressure source 70 passes through an air filter 72 into a valve manifold 74, which is operatively connected to a pulse train generator 76. Pressurized air from valve manifold 74 is directed to pressure regulation controls 78,80,82,84 in a displacement actuator air piston cylinder 86 connected to actuator rod 32 and a displacement actuator air piston cylinder 88 connected to pressure. control rod 46. The latter air piston provides the force necessary to displace the pressure control rod 46 by utilizing the mechanical advantage of converting a low pressure on a large piston area into a high pressure on a small piston area. Actuator rod 32 and pressure control rod 46 each engage external loose bellows 47, 49, which provide a separation of the internal sterile environment of the bioreactor from the outside. Dependent upon the instructions from the pulse train generator 76, the displacement of the compression loading platen in the bioreactor is increased or decreased, and the hydrostatic pressure is increased or decreased.

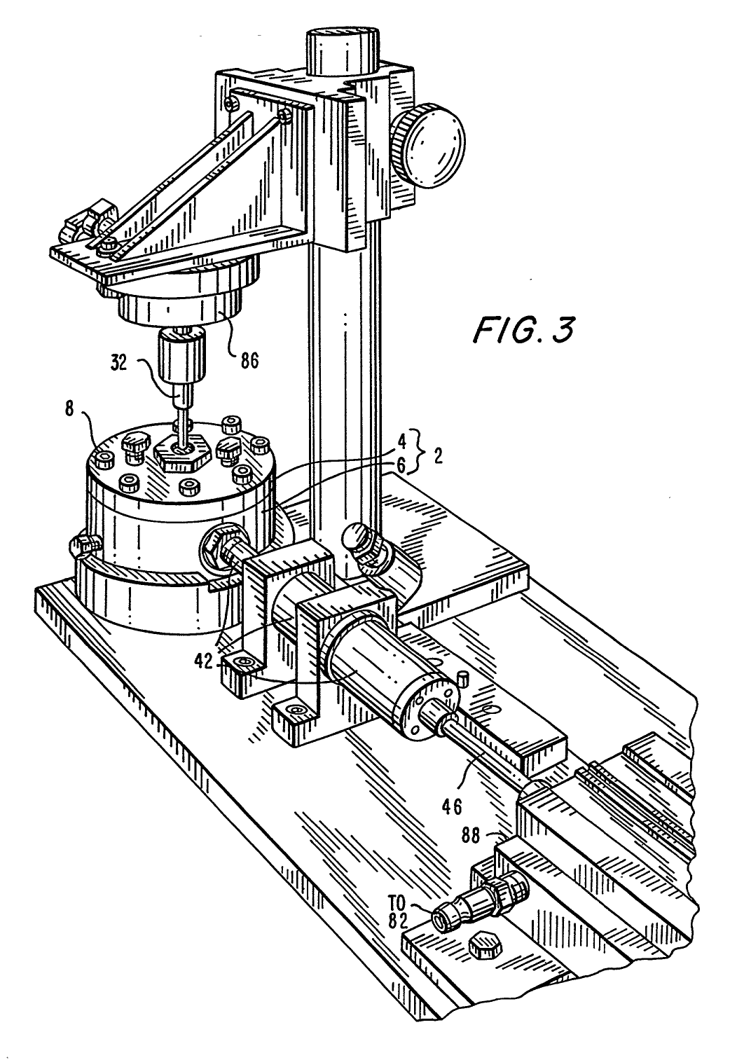

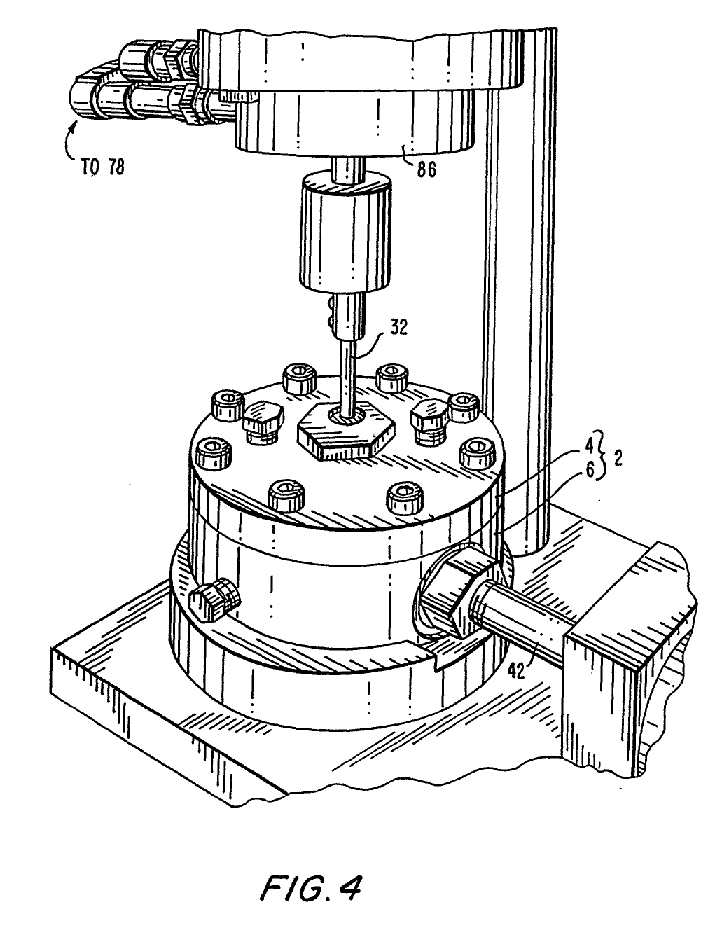

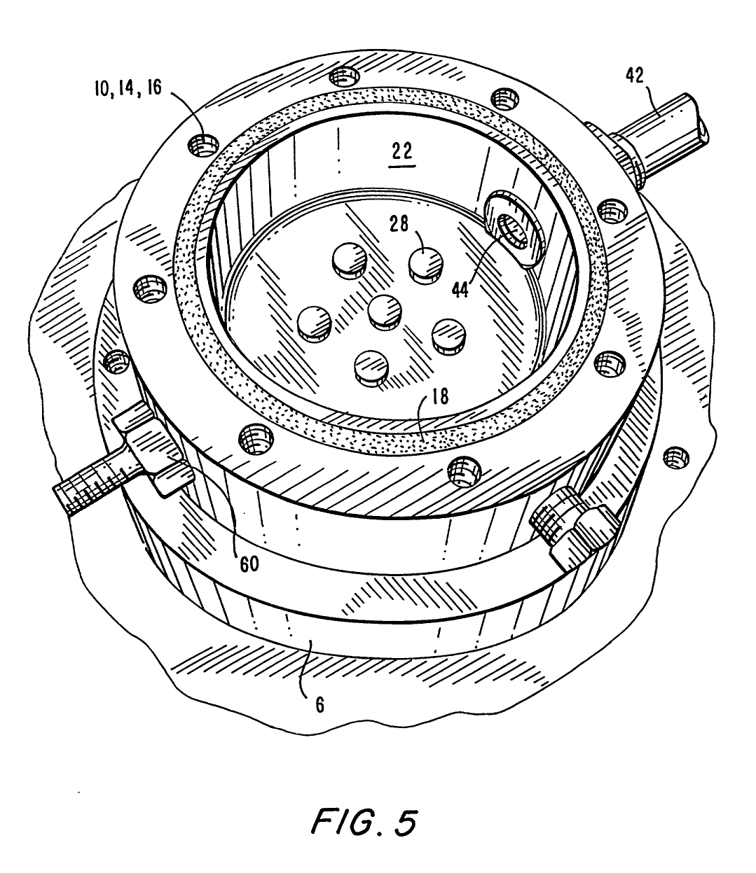

FIG. 3 is a perspective view of one embodiment of the bioreactor 2 with a compressive strain (deformational loading) air cylinder 86 and a hydrostatic pressure air cylinder assembly 88. A closer view of bioreactor 2 is provided in FIG. 4, which clearly displays compressive strain air cylinder assembly 86 and displacement actuator rod 32 that collectively form the displacement actuator device. FIG. 5 is a view of the interior chamber 22 of bioreactor 2, which is the interior of lower member 6 in which the vessel cap 4 (not shown) is secured with circular O-ring 18 and threaded screws into threaded openings 10,14,16. A partial view of piston chamber lumen 44 of hydraulic pressure assembly 42 for which a hydraulic pressure control rod or piston is extended within to increase the pressure in chamber 22 can be seen. A pressure transducer 60 is used to monitor pressure development within chamber 22. Chondrocyte-seeded agarose disks 28 have been positioned within chamber 22. During normal functioning of the bioreactor, chamber 22 would be completely filled with cell culture medium supplemented with appropriate factors (such as nutrients, growth factors, buffers, etc.).

A typical loading regimen for the cell-seeded agarose disks consists of applying cyclical hydrostatic pressure with an amplitude of 2 MPa and/or deformational loading with an amplitude of 10%, at a frequency of 1 Hz. The time-course of dynamic loading consists of three consecutive 1-hour-on, 1-hour-off cycles per day, for 5 days per week, for 8 weeks.

The objective of the above example is to provide a physiologic loading environment for the chondrocyte-seeded agarose disks to promote growth of functional hyaline cartilage. One advantage of agarose over other scaffold materials is that it can sustain mechanical loading at physiologic strains without permanent deformation. Together the biocompatibility and mechanical properties of agarose make it possible to apply load to chondrocyte-seeded agarose cultures immediately upon seeding of cells. This allows for assessment of the effects of mechanical loads during the initial stages of tissue development.

In the unconfined compression configuration described above, the cell-seeded agarose disk is loaded between impermeable smooth loading platens and is free to expand laterally (i.e., in the radial direction). The interstitial fluid hydrostatic pressure and the scaffold compressive strain along the axial direction of the cylindrical disk are uniform through the thickness of the sample, and there is no fluid flow relative to the solid matrix along the axial loading direction. Similarly, at physiologic loading rates (0.1-5 Hz), the hydrostatic pressure and tensile radial and circumferential strains are uniform from the center almost to the periphery of the sample, with pressure, strain and fluid flow gradients occurring only in a narrow region near the sample edges. Thus, overall, the configuration of unconfined compression produces more uniform mechanical signals throughout a cylindrical sample than that of confined compression, which is more suitable for tissue engineering purposes. Furthermore, the uniformity of the interstitial fluid pressure through the depth of the sample is more physiologic; unconfined compression produces both compressive strains (along the axial direction) and tensile strains (along the radial and circumferential directions), which also represents a more physiologic loading environment than confined compression, as suggested by analyses of contacting cartilage layers. Finally, unconfined compression can produce tissue strains with negligible change in tissue volume (since the disk can expand laterally when compressed axially), while confined compression is always accompanied by loss of tissue volume due to water efflux; in vivo measurements of cartilage volumetric changes have been shown to be small (6%) even following strenuous loading. For all these reasons, the loading configuration adopted for the above example is that of unconfined compression.

Because of the differences in material properties between agarose gels and normal articular cartilage, applying up to 10% compression on agarose disks will produce hydrostatic pressures which are negligible compared to the desired physiological levels; thus, it is necessary to externally pressurize the agarose gels to provide the desired physiological loading environment for the chondrocyte-seeded scaffolds. However, as a cartilage-like matrix is produced by the chondrocytes over time, the magnitude of interstitial fluid pressure resulting from the imposed deformation of the agarose gels may increase, possibly as high as 1-2 MPa. Under these circumstances, the chondrocytes would be subjected to the compounded effect of interstitial fluid and bathing solution pressurization. Finally, the loading rate of 1 Hz suggested above is motivated by the need to produce physiological loading conditions. It is reasonable to expect that human joints can be comfortably subjected to activities of moderate loading at a nearly cyclical rate of 1 Hz, continuously for 30 minutes or longer (e.g., going on a walk—for loading of the lower extremities—or writing with pen on paper—for loading of the finger and thumb joints).

Preparation of chondrocyte-seeded agarose scaffolds

Cylindrical disks consisting of chondrocytes suspended in agarose can be prepared as follows. Articular cartilage is harvested from the carpo-metacarpal joint of freshly killed 4-6 month old bovine calves obtained from a local abattoir and rinsed in Dulbecco's Modified Essential Medium (DMEM) supplemented with 10% FBS, amino acids (0.5× minimal essential amino acids, 1X non-essential amino acids), buffering agents (10 mM Hepes, 10 mM sodium bicarbonate, 10 mM TES, 10 mM BES), and antibiotics (100 U/ml penicillin, 100 μg/ml streptomycin). The cartilage chunks are digested with 50 mg of bovine testicular hyaluronidase type I-S (Sigma Chemical Company, St. Louis, Mo.) in 100 ml of DMEM for 30 minutes at 37° C. After removal of the hyaluronidase solution, the cartilage specimens are digested at 37° C. overnight with 50 mg of clostridial collagenase type II (Sigma) in 100 ml of DMEM. The cell suspension will then be sedimented in a benchtop clinical centrifuge at 4° C. for 5 minutes. After rinsing the pellets, the cells are finally resuspended in 10 ml of DMEM, and viable cells are counted using a hemacytometer and trypan blue exclusion.

For the preparation of chondrocyte/agarose constructs, one volume of chondrocyte suspension (2×10⁷ cells/ml) is mixed with an equal volume of 4% molten Type VII agarose (Sigma) in Hank's balanced salt solution (HBSS) at 37° C. to yield a final cell concentration of 1×10⁷ cells/ml in 2% agarose. After mixing, the chondrocyte/agarose mixture is poured into sterile 16 cm ×20 cm molds consisting of two glass plates separated by 3-mm spacers. The molds areincubated at 4° C. for 10 min to allow the agarose to gel. Cylindrical disks of 10-mm diameter are then cored from the chondrocyte/agarose slabs with a 10-mm trephine, rinsed twice in DMEM and cultured as described below.

Culturing of chondrocyte/agarose contructs

Chondrocyte/agarose disks are maintained in culture for up to 6 weeks (42 days), with daily change of growth medium. The growth medium consists of DMEM supplemented as indicated above. The medium is also supplemented with 50 μg ascorbate/ml. Disks are grown in the bioreactor which is placed in an incubator, preferably at 37° C. As loading is carried out every day, cell-laden disks are left in the vessel base for overnight culture. Fresh media is introduced into the bioreactor on a daily basis using access ports.

Design and production of scaffold shapes and bioreactor loading platens

As taught in U.S. Pat. No. 6,126,690, incorporated herein by reference, for the description of the fabrication of a joint prosthesis, the anatomic shape of the loading platen and scaffold can be based upon obtaining imaging data (e.g., stereophotogram-metry, magnetic resonance imaging, computed tomography) of a patient's healthy contralateral joint surfaces and optionally modifying the imaged data of the patient's healthy contralateral joint surfaces to provide a more functional surface topography. Alternatively, the anatomic shape of the loading platen and scaffold can be based on a database of a plurality of joint surface archetypes acquired through measurement of a plurality of joint surfaces, said plurality of joint surface archetypes being cross-referenced by parameters including dimensions of bone associated with joint surface, the weight of a person from whom the measurement is being taken, the sex of the person from whom the measurement is being taken, the race of the person from whom the measurement is being taken, and the height of the person from whom the measurement is being taken, input means for receiving a plurality of parameters exhibited by the patient, a microprocessor connected to said memory means for selecting one of said plurality of joint surface archetypes whose parameters most closely resemble a corresponding plurality of parameters exhibited by the patient, by said microprocessor for fabricating the joint prosthesis to resemble the selected articular joint surface archetype. The imaged data of the articular topography can then be converted into a three-dimensional surface contour using commercially available computer-aided design software. These contours can be employed to create a solid computer model from which physical molds can be generated using a technique for three-dimensional fabrication (such as computerized numerical control machine tools, rapid prototype machine, stereolithography). These molds then serve to create a scaffold having the articular topography of the desired imaged data as well as loading platens that mate congruently with the scaffold surface

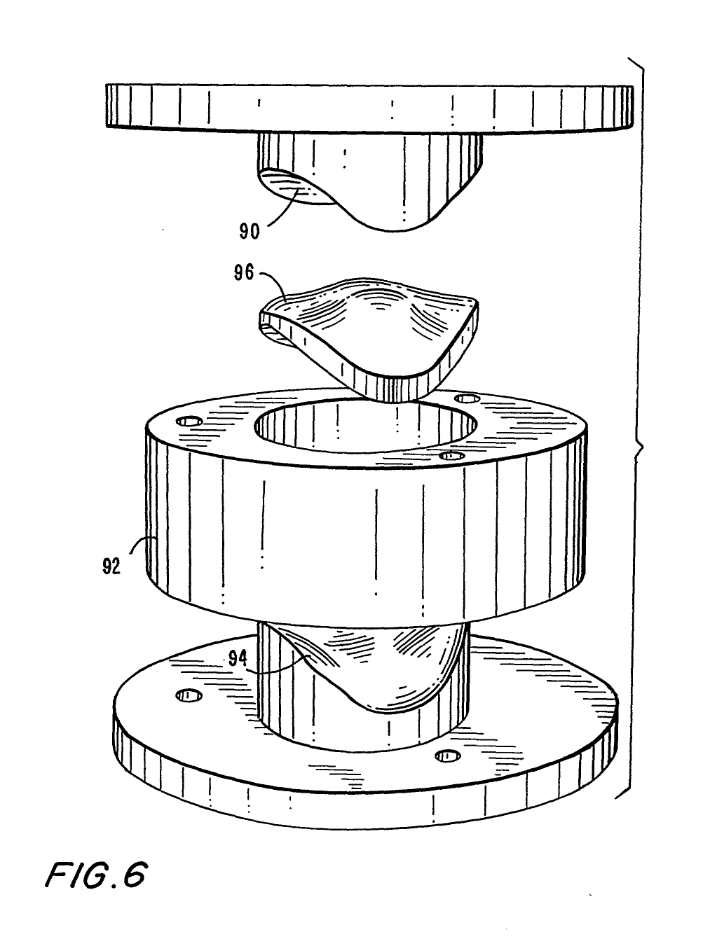

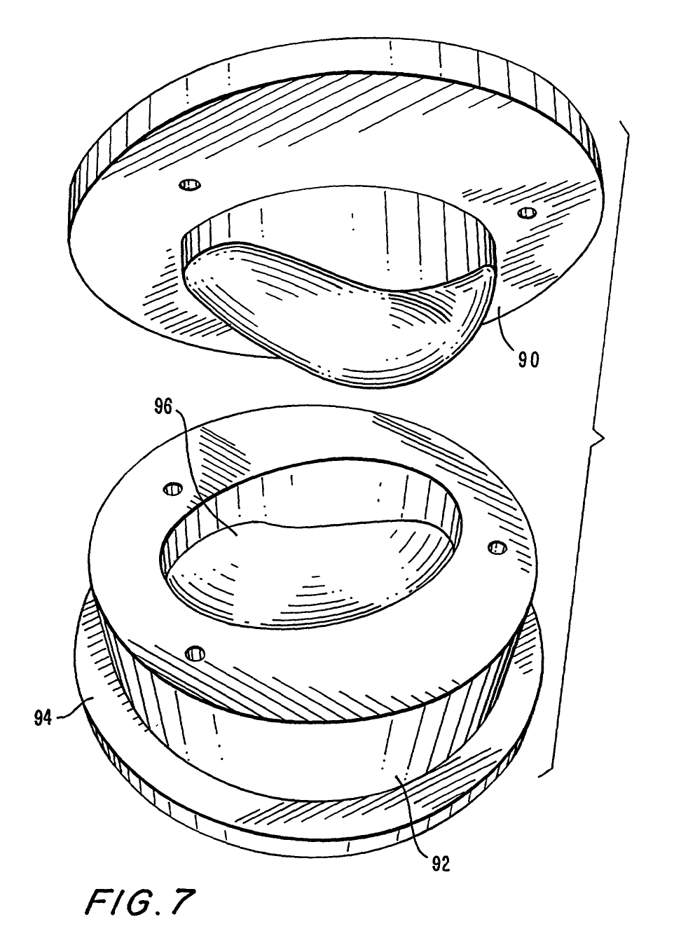

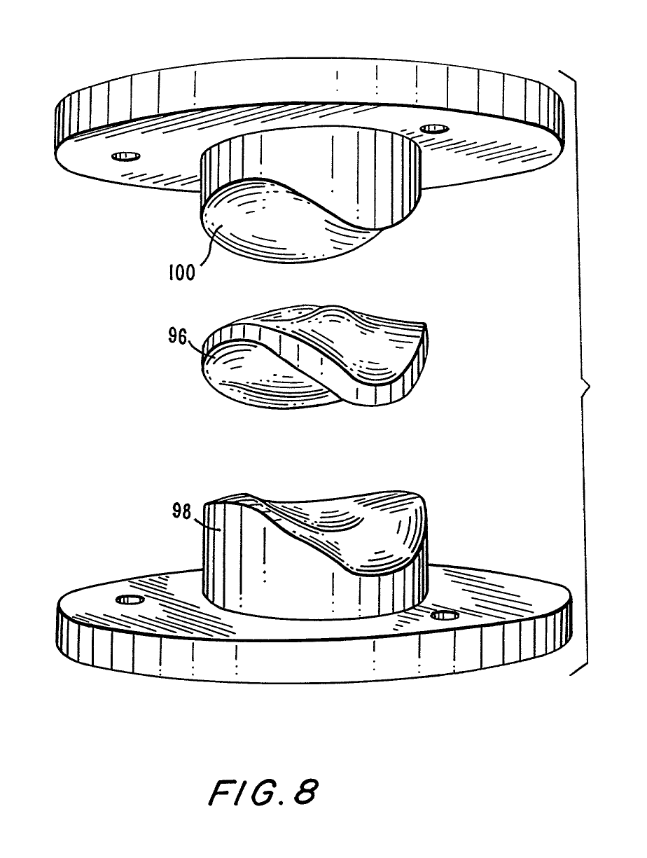

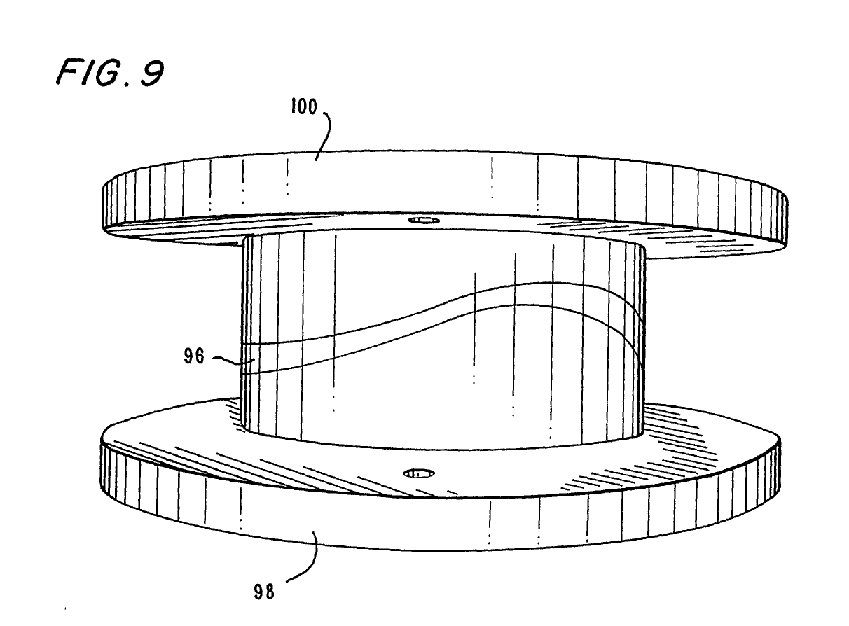

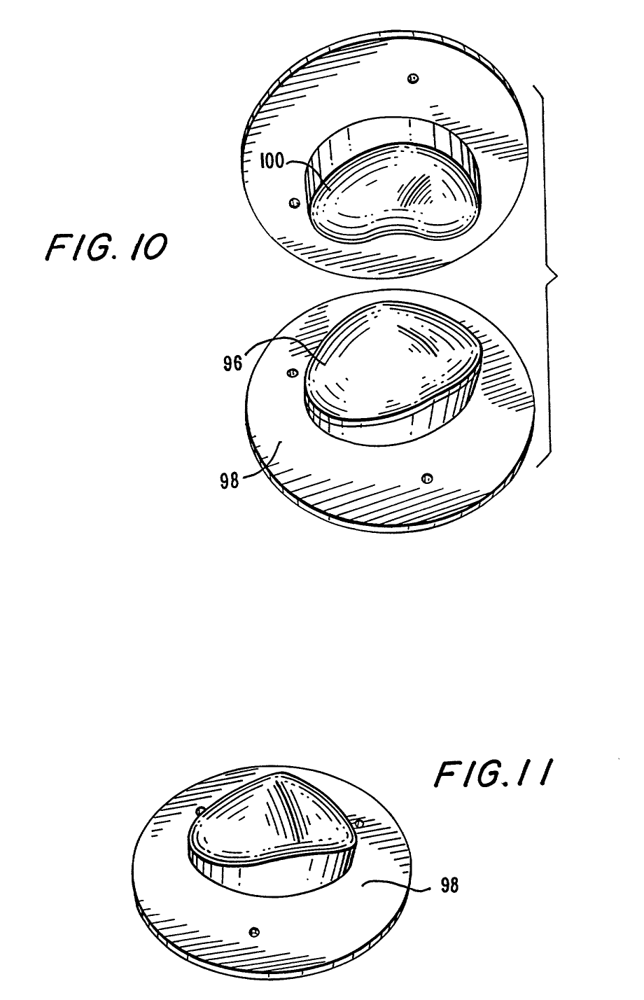

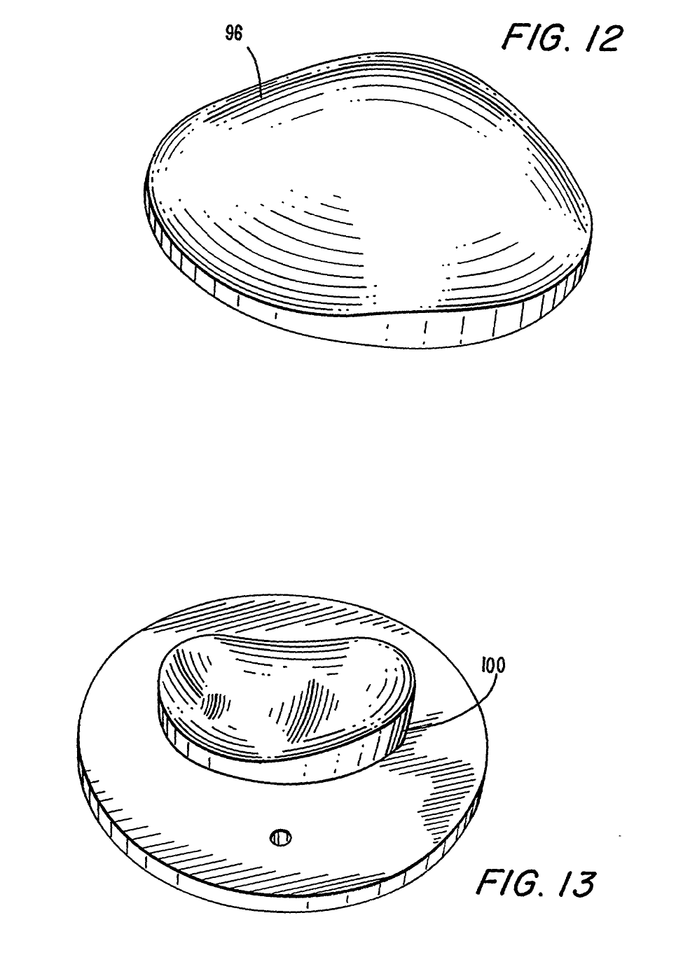

To illustrate the methodology described herein, FIGS. 6 to 13 depict the creation of an agarose scaffold construct having the articular layer topography of a human patella that has been generated using a mold fabricated using rapid prototype machining. A computer-aided design drawing of the mold and scaffold construct are shown in FIG. 6, whereas a rapid prototype of this mold containing the agarose scaffold construct 96 is shown in FIG. 7. Two halves of the mold (each having the specified articular topography of the articular surface 90 and subchondral bone surface 94 are separated by a spacer ring 92 that defines the thickness of the scaffold construct and serves to create an enclosed volume having the shape of the desired construct. In one embodiment, melted 2% agarose containing chondrocytes or other progenitor cells has been poured into the mold and permitted to cool, resulting with the creation of a three-dimensional agarose scaffold construct having the surface topography of the desired articular layer (FIG. 7, 96). To illustrate how the scaffold can be loaded with platens having the same articular surface topography, FIG. 8 depicts a computer-aided design drawing of the scaffold construct 96 when it has been seated between two congruent loading platens 98,100, whereas FIG. 9 depicts the scaffold construct 96 when it has been seated between two congruent loading platens 98,100 constructed of ABS plastic from the rapid prototype machine. FIG. 10 depicts the agarose construct seated on the lower platen 98 conforming to the subchondral bone surface, with the top platen 100, conforming to the articular surface, removed and in the background. FIG. 11 depicts the lower loading platen 98, FIG. 12 depicts the three dimensional agarose construct 96 created from the mold 90, 92, 94, and FIG. 13 depicts the upper loading platen 100. The preferred embodiment for the mold and loading platen material would be one that is sterilizable, rigid and machineable (such as stainless steel, polysulfone).

In another embodiment of the invention, the loading platen reproducing the subchondral bone surface of the anatomic articular layer is replaced with a porous osteoconductive and/or osteoinductive anatomically shaped substrate which similarly reproduces the subchondral bone surface. A solution, such as melted 2% agarose, containing chondrocyte or progenitor cells is then poured over and into the porous substrate. This anatomically shaped substrate, optionally modified, will serve subsequently as a part of the scaffold construct to promote bony integration in vivo. Bone cells or bone progenitor cells can be seeded into or onto the bony substrate.

In yet another embodiment of the invention, the molds described herein are used to create scaffold constructs from a variety of biomaterials, having the anatomic shape of a desired articular layer surface, which are then seeded with chondrocytes or progenitor cells and then subsequently subjected to physiologic loading using the bioreactor with loading platens that are conforming to the shape of the scaffold construct.

In a further emodiment of this invention, the aforementioned scaffold construct can be attached (such as with a biocompatible adhesive, suturing etc.) to a bony substrate (osteoconductive and/or osteoinductive) that forms the loading platen facing the subchondral side of the anatomic articular layer. This loading platen, optionally modified, will serve subsequently as a part of the scaffold construct to promote bony integration in vivo. Bone cells or bone cell progenitor cells can be seeded into or onto the bony substrate.

The preceding specific embodiments are illustrative of the practice of the invention. It is to be understood, however, that other expedients known to those skilled in the art or disclosed herein, may be employed without departing from the spirit of the invention or the scope of the appended claims.

What is claimed is:

1. A bioreactor for producing functional cartilaginous tissue from a cell-seeded scaffold or a cell-seeded scaffold integrated with an osteoconductive and/or osteoinductive substrate, comprising:
(a) a growth chamber, and
(b) means for applying hydrostatic and/or deformational loading to the cell-seeded scaffold or cell-seeded scaffold integrated with an osteoconductive and/or osteoinductive substrate.

2. The bioreactor of claim 1, wherein the scaffold is bioresorbable.

3. The bioreactor of claim 1, wherein the scaffold is biocompatible.

4. The bioreactor of claim 1, wherein the scaffold is biodegradable.

5. The bioreactor of claim 1, wherein the scaffold is non-biodegradable.

6. The bioreactor of claim 1, wherein means (b) applies intermittent cyclical hydrostatic fluid pressurization.

7. The bioreactor of claim 6, wherein the fluid pressurization is from about 0 to about 18 MPa.

8. The bioreactor of claim 7, wherein the fluid pressurization is from about 0 to about 6 MPa.

9. The bioreactor of claim 6, wherein the cyclical frequency is from about 0 to about 5 Hz.

10. The bioreactor of claim 9, wherein the cyclical frequency is from about 0.1 to about 2 Hz.

11. The bioreactor of claim 1, wherein the fluid pressurization is applied for from about 0.5 to about 18 hours per day.

12. The bioreactor of claim 11, wherein the fluid pressurization is applied for from about 2 to about 6 hours per day.

13. The bioreactor of claim 1, wherein means (b) applies intermittent cyclical deformational loading.

14. The bioreactor of claim 13, wherein the deformational loading is from about 0 to about 50%, based upon the thickness of the cell-seeded scaffold.

15. The bioreactor of claim 14, wherein the deformational loading is from about 0 to about 20%.

16. The bioreactor of claim 13, wherein the cyclical frequency is from about 0 to about 5 Hz.

17. The bioreactor of claim 16, wherein the cyclical frequency is from about 0.1 to about 2 Hz.

18. The bioreactor of claim 13, wherein the deformational loading is from about 0.5 to about 18 hours per day.

19. The bioreactor of claim 18, wherein the deformational loading is from about 2 to about 6 hours per day.

20. The bioreactor of claim 1, wherein means (b) applies intermittent cyclical hydrostatic fluid pressurization and intermittent cyclical deformational loading.

21. The bioreactor of claim 20, wherein the amplitude of the hydrostatic pressure and the amplitude of the deformational loading are modified over time as matrix elaboration proceeds.

22. The bioreactor of claim 1, wherein the resulting tissue comprises hyaline cartilage.

23. The bioreactor of claim 1, wherein the resulting tissue comprises hyaline cartilage and a osteoconductive and/or osteoinductive substrate.

24. The bioreactor of claim 1, wherein the resulting tissue comprises elastic cartilage.

25. The bioreactor of claim 1, wherein the resulting tissue comprises fibrocartilage.

26. The bioreactor of claim 1 which comprises means for producing tissue in desired shapes.

27. The bioreactor of claim 26, wherein the shaped tissue conforms to a body part, a prosthesis, a cosmetic implant, or a defect to be filled.

28. The bioreactor of claim 1, wherein the loading platens which produce deformational loading conform to a body part, a prosthesis, a cosmetic implant, or a defect to be filled.

29. A method for producing functional cartilaginous tissue from a cell-seeded scaffold or a cell-seeded scaffold integrated with an osteoconductive and/or osteoinductive substrate, said method comprising the steps of:
(a) inoculating chondrocytes or chondroprogenitors into a scaffold or a scaffold integrated with an osteoconductive and/or osteoinductive substrate;
(b) placing cell-seeded scaffold or cell-seeded scaffold integrated with an osteoconductive and/or osteoinductive substrate into a bioreactor
(c) filling said bioreactor with liquid growth medium.
(d) applying hydrostatic pressurization and/or deformational loading to the cell-seeded scaffold or cell-seeded scaffold integrated with an osteoconductive and/or osteoinductive substrate; and
(e) culturing said stressed cell-seeded scaffold or cell-seeded scaffold integrated with an osteoinductive substrate for a time sufficient to produce functional cartilaginous tissue.

30. The method of claim 29, wherein the bioreactor is the bioreactor of claim 1.

31. The method of claim 29, wherein the scaffold is biocompatible.

32. The method of claim 29, wherein the scaffold is biodegradable.

33. The method of claim 29, wherein the scaffold is non-biodegradable.

34. The method of claim 29, wherein the scaffold is bioresorbable.

35. The method of claim 29, wherein said stressed cells:
(a) display enhanced maintenance of a chondrocyte phenotype; and
(b) produce a functional cartilaginous matrix.

36. The method of claim 29, wherein hydrostatic pressurization is applied by means comprising a reservoir, a pump, and tubing interconnecting said growth chamber, said reservoir, and said pump, so as to allow pressurization of liquid growth medium from said reservoir, in response to force applied by said pump.

37. The method of claim 36, wherein said pump comprises a piston and chamber.

38. The method of claim 29, wherein in step (d) intermittent cyclical hydrostatic fluid pressurization is applied.

39. The method of claim 38, wherein the fluid pressurization is from about 0 to about 18 MPa.

40. The method of claim 39, wherein the fluid pressurization is from about 0 to about 6 MPa.

41. The method of claim 38, wherein the cyclical frequency is from about 0 to about 5 Hz.

42. The method of claim 41, wherein the cyclical frequency is from about 0.1 to about 2 Hz.

43. The method of claim 29, wherein the fluid pressurization is applied for from about about 0.5 to about 18 hours per day.

44. The method of claim 43, wherein the fluid pressurization is applied for from about 2 to about 6 hours per day.

45. The method of claim 29, wherein in step (d) intermittent cyclical deformational loading is applied.

46. The method of claim 45, wherein the deformational loading is from about 0 to about 50%, based upon the thickness of the cell-seeded scaffold.

47. The method of claim 46, wherein the deformational loading is from about 0 to about 20%.

48. The method of claim 45, wherein the cyclical frequency is from about 0 to about 5 Hz.

49. The method of claim 48, wherein the cyclical frequency is from about 0.1 to about 2 hz.

50. The method of claim 45, wherein the deformational loading is from about 0.5 to about 18 hours per day.

51. The method of claim 50, wherein the deformational loading is from about 2 to about 6 hours per day.

52. The method of claim 29, wherein in step (d) intermittent cyclical hydrostatic fluid pressurization and intermittent cyclical deformational loading are applied.

53. The method of claim 52, wherein the amplitude of the hydrostatic pressure and the amplitude of the deformational loading are modified over time as matrix elaboration proceeds.

54. The method of claim 29, wherein the resulting tissue comprises hyaline cartilage.

55. The method of claim 29, wherein the resulting tissue comprises hyaline cartilage and osteoinductive substrate.

56. The method of claim 29, wherein the resulting tissue comprises elastic cartilage.

57. The method of claim 29, wherein the resulting tissue comprises fibrocartilage.

58. The method of claim 29 wherein the bioreactor comprises means for producing tissue in desired shapes.

59. The bioreactor of claim 29 where the loading platens which produce deformational loading conform to a body part, a prosthesis, a cosmetic implant, or a defect to be filled.

60. The method of claim 59, wherein the shaped tissue conforms to a body part, a prosthesis, a cosmetic implant, or a defect to be filled.
